(19) United States
(12) Patent Application Publication
(10) Pub. No.: US 2009/0148883 A1
(43) Pub. Date: Jun. 11, 2009

(54) BIOMARKER FOR ASSESSING RESPONSE TO FMS TREATMENT

(76) Inventor: Carl L. Manthey, Downingtown, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/261,558

(22) Filed: Oct. 30, 2008

Related U.S. Application Data
(60) Provisional application No. 60/984,122, filed on Oct. 31, 2007.

(51) Int. Cl. C12Q 1/02

(52) U.S. Cl. 435/29

(57) ABSTRACT

A biomarker that correlates to treatment with drugs that inhibit FMS is disclosed. This biomarker has been shown to have utility in assessing response to the compounds. The plasma level of the biomarker is increased upon treatment with FMS inhibitor compounds, thus indicating that this biomarker is involved in FMS activity.

### CROSS REFERENCE TO RELATED APPLICATIONS

This application claims priority to Application No. 60/984,122, filed on Oct. 31, 2007, the entire contents of which are incorporated by reference herein.

### FIELD OF THE INVENTION

The present invention relates generally to the field of pharmacodynamics, and more specifically to materials, methods and procedures to determine drug sensitivity in patients, including in patients with cancer. This invention aids in treating diseases and disorders based on patient response at a molecular level.

### BACKGROUND OF THE INVENTION

A number of drugs that reduce or inhibit the activity of FMS are currently being developed. See, for example, U.S. Patent Publication Nos. 2006-0148812-A1 and 2006-0189623-A1, the entire contents of which are incorporated herein by reference. Predictive markers are needed to accurately foretell a patient's response to such drugs in the clinic. Such markers would facilitate the individualization of therapy for each patient.

The present invention is directed to the identification of a biomarker that can better predict a patient's sensitivity to treatment or therapy with drugs that reduce or inhibit FMS. The association of a patient's response to drug treatment with this marker can open up new opportunities for drug development in non-responding patients, or distinguish a drug's indication among other treatment choices because of higher confidence in the efficacy. Further, the pre-selection of patients who are likely to respond well to a drug or combination therapy may reduce the number of patients needed in a clinical study or accelerate the time needed to complete a clinical development program (M. Cockett et al., 2000, Current Opinion in Biotechnology, 11:602-609). A major goal of research is to identify markers that accurately predict a given patient's response to drugs in the clinic; such individualized assessment may greatly facilitate personalized treatment. An approach of this nature is particularly needed in cancer treatment and therapy, where commonly used drugs are ineffective in many patients, and side effects are frequent. The ability to predict drug sensitivity in patients is particularly challenging because drug responses reflect both the properties intrinsic to the target cells and also a host's metabolic properties.

Needed in the art are materials, methods and procedures to determine drug sensitivity in patients in order to treat diseases and disorders, particularly cancers, based on patient response at a molecular level. The present invention involves the identification of a biomarker that correlates with drug sensitivity to drugs that reduce or inhibit FMS. The presently described identification of marker can be extended to clinical situations in which the marker is used to predict responses to drugs that reduce or inhibit FMS.

Bartocci et al., Proc. Natl. Acad. Sci. USA, 84:6179-6183 (1987), discloses that CSF-1 is cleared from the circulation of mice by liver macrophages. The clearance is apparently by CSF-1 receptor-mediated endocytosis and intracellular destruction.

Carlberg et al., EMBO Journal, 10(4):877-883 (1991), discloses that CSF-1 binding induced internalization and degradation of the receptor and the rate of degradation of a kinase-defective mutant receptor was reduced but not eliminated.

Xu-Ming et al., Blood, 99(1):111-120 (2002), discloses that inactivation of mouse CSF-1 receptor gene resulted in a 20-fold elevation in circulating CSF-1.

Irvine et al., FASEB J., 20:1315-1322 (2006), discloses that CYC10268 is an inhibitor of the CSF-1 receptor that failed to inhibit CSF-1-induced receptor depletion after twenty minutes of CSF-1 exposure.

### SUMMARY OF THE INVENTION

The present invention is related to the identification that increased serum or plasma levels of CSF-1 is correlated with inhibition of the FMS receptor. This “marker” shows utility in predicting a host's response to a drug and/or drug treatment.

It is an aspect of the invention to provide a method of monitoring the treatment of a patient having a disease treatable by a drug that modulates FMS. This can be accomplished by comparing the level of CSF-1 in serum or plasma from a patient prior to treatment with a drug that inhibits FMS activity and again following treatment with the drug. Thus, if a patient's response becomes one that is sensitive to treatment by a FMS inhibitor compound, based on a correlation of an observed increase in serum or plasma CSF-1, the patient's treatment prognosis can be qualified as favorable and treatment can continue. Also, if after treatment with a drug, the patient's serum or plasma level of CSF-1 does not increase, this can serve as an indicator that the current treatment should be modified, changed, or even discontinued. Such a monitoring process can indicate success or failure of a patient's treatment with a drug, and the monitoring processes can be repeated as necessary or desired.

### DESCRIPTION OF THE FIGURES

FIG. 1 is a linear-linear plot showing clearance of CSF-1 by bone marrow derived macrophages (BMDM) in vitro in the presence and absence of COMPOUND 1 (all data except vehicle and circle) and another compound (circle). The structure of COMPOUND 1 is reproduced below:

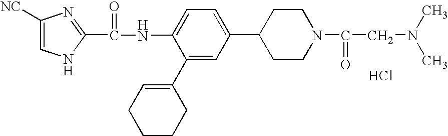

FIG. 2 is a log-linear plot with linear regression analysis showing clearance of CSF-1 by BMDM in vitro in the presence and absence of COMPOUND 1.

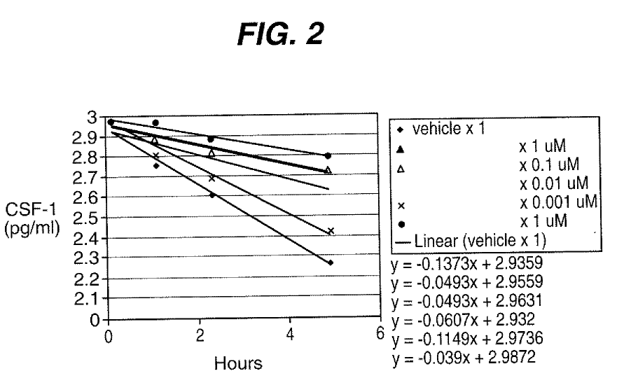

FIGS. 3A and 3B show the effects of COMPOUND 2 (5 mg/kg and 15 mg/kg) and COMPOUND 1 (40 mg/kg) on MMCSF-1 Levels in Plasma. The structure of COMPOUND 2 is reproduced below:

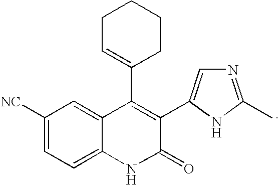

COMPOUND 2 and its used are disclosed in U.S. Application No. 20050049274A1.

FIG. 4 shows the effects of COMPOUND 2 (5 mg/kg and 15 mg/kg) and COMPOUND 1 (40 mg/kg) on Macrophage Content of the Uterus

### DETAILED DESCRIPTION OF THE INVENTION

All publications cited herein are hereby incorporated by reference. Unless defined otherwise, all technical and scientific terms used herein have the same meaning as commonly understood to one of ordinary skill in the art to which this invention pertains.

### Definitions

As used herein, the terms “comprising”, “containing”, “having” and “including” are used in their open, non-limiting sense.

A “biological sample” as used herein refers to a sample containing or consisting of cells or tissue matter, such as cells or biological fluids isolated from a subject. The “subject” can be a mammal, such as a rat, a mouse, a monkey, or a human, that has been the object of treatment, observation or experiment. Examples of biological samples include, for example, sputum, blood, blood cells (e.g., white blood cells), amniotic fluid, plasma, serum, semen, saliva, bone marrow, tissue or fine-needle biopsy samples, urine, peritoneal fluid, pleural fluid, and cell cultures. Biological samples may also include sections of tissues such as frozen sections taken for histological purposes. A test biological sample is the biological sample that has been the object of analysis, monitoring, or observation. A control biological sample can be either a positive or a negative control for the test biological sample. Often, the control biological sample contains the same type of tissues, cells and/or biological fluids of interest as that of the test biological sample. In particular embodiments, the biological sample is a “clinical sample,” which is a sample derived from a human patient.

A “cell” refers to at least one cell or a plurality of cells appropriate for the sensitivity of the detection method. The cell can be present in a cultivated cell culture. The cell can also be present in its natural environment, such as a biological tissue or fluid. Cells suitable for the present invention may be bacterial, but are preferably eukaryotic, and are most preferably mammalian.

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by the codons of genes may also be included in a polypeptide.

An “isolated protein” is one that is substantially separated from at least one of the other proteins present in the natural source of the protein, or is substantially free of at least one of the chemical precursors or other chemicals when the protein is chemically synthesized. A protein is “substantially separated from” or “substantially free of” other protein(s) or other chemical(s) in preparations of the protein when there is less than about 30%, 20%, 10%, or 5% (by dry weight) of the other protein(s) or the other chemical(s) (also referred to herein as a “contaminating protein” or a “contaminating chemical”).

Isolated proteins can have several different physical forms. The isolated protein can exist as a full-length nascent or unprocessed polypeptide, or as a partially processed polypeptide or as a combination of processed polypeptides. The full-length nascent polypeptide can be postranslationally modified by specific proteolytic cleavage events that result in the formation of fragments of the full-length nascent polypeptide. A fragment, or physical association of fragments can have the biological activity associated with the full-length polypeptide; however, the degree of biological activity associated with individual fragments can vary.

An isolated polypeptide can be a non-naturally occurring polypeptide. For example, an “isolated polypeptide” can be a “hybrid polypeptide.” An “isolated polypeptide” can also be a polypeptide derived from a naturally occurring polypeptide by additions or deletions or substitutions of amino acids. An isolated polypeptide can also be a “purified polypeptide” which is used herein to mean a specified polypeptide in a substantially homogeneous preparation substantially free of other cellular components, other polypeptides, viral materials, or culture medium, or when the polypeptide is chemically synthesized, chemical precursors or by-products associated with the chemical synthesis. A “purified polypeptide” can be obtained from natural or recombinant host cells by standard purification techniques, or by chemical synthesis, as will be apparent to skilled artisans.

The present invention describes the identification that serum or platelet levels of CSF-1 serves as a useful molecular tool for predicting a response to drugs that affect FMS activity via direct or indirect inhibition or antagonism of the FMS function or activity.

Also provided by the present invention are monitoring assays to monitor the progress of drug treatment involving drugs that interact with or inhibit FMS activity. Such in vitro assays are capable of monitoring the treatment of a patient having a disease treatable by a drug that modulates or interacts with FMS by comparing serum or plasma levels of CSF-1 prior to treatment with a drug that inhibits FMS activity and again following treatment with the drug. Isolated cells from the patient are assayed to determine the level of CSF-1 before and after exposure to a drug, preferably a FMS inhibitor, to determine if a change of the has occurred so as to warrant treatment with another drug, or whether current treatment should be discontinued.

In another embodiment, the human FMS biomarker can be used for screening therapeutic drugs in a variety of drug screening techniques.

The term “drug” is used herein to refer to a substance that potentially can be used as a medication or in the preparation of a medication. Essentially any chemical compound can be employed as a drug in the assays according to the present invention. Compounds tested can be any small chemical compound, or biological entity (e.g., amino acid chain, protein, sugar, nucleic acid, or lipid). Test compounds are typically small chemical molecules and peptides. Generally, the compounds used as potential modulators can be dissolved in aqueous or organic (e.g., DMSO-based) solutions. The assays are designed to screen large chemical libraries by automating the assay steps and providing compounds from any convenient source. Assays are typically run in parallel, for example, in microtiter formats on microtiter plates in robotic assays. There are many suppliers of chemical compounds, including, for example, Sigma (St. Louis, Mo.), Aldrich (St. Louis, Mo.), Sigma-Aldrich (St. Louis, Mo.), Fluka Chemika-Biochemica Analytika (Buchs, Switzerland). Also, compounds can be synthesized by methods known in the art.

### EXAMPLES

The Examples herein are meant to exemplify the various aspects of carrying out the invention and are not intended to limit the scope of the invention in any way.

### Example 1

Clearance of CSF-1 by Bone Marrow Derived Macrophages In Vitro is Inhibited by COMPOUND 1

COMPOUND 1 was provided as a 10 mM stock in dimethyl sulfoxide (DMSO).Recombinant mouse CSF-1 (416 mL 050) and mouse CSF-1 ELISA (MNC00) were both purchased from R&D Systems Inc., (Minneapolis, Minn.).

Mouse bone marrow-derived macrophage (BMDM) were prepared and were plated into 12 well plates in the presence of culture media (αMEM containing 10% FCS and glutamine PenStrep) and 25 ng/mL CSF-1 for three days until approximately 60% confluency was achieved. The cultures were then washed twice with media without CSF-1, and cultured for 2 hrs in the absence of CSF-1 to allow the macrophages to consume CSF-1 that may have been residual to the wells. The cultures were next adjusted to contain CSF-1 and COMPOUND 1 as indicated in Table 1 (Experimental Design) and incubation was continued at 37° C. and 5% CO₂. Some wells did not have cells and served as controls for device binding and CSF-1 stability. At timed-intervals (0, 1.1, 2.3, 4.9, 10.3, 19.9, 29, and 44 hours) 50 μl of conditioned media were removed from each well and saved under refrigeration. After harvest of the last time point, all samples were subjected to CSF-1 ELISA.

TABLE 1
Experimental Design

| | Items added to wells | | |
|---|---|---|---|
| Well # | BMDM | CSF-1 | COMPOUND 1 |
| 1 | NO | 1 ng/mL | None |
| 2 | NO | none | None |
| 3 | YES | none | None |
| 4 | YES | 1 ng/mL | None |
| 5 | YES | 1 ng/mL | 0.001 μM |
| 6 | YES | 1 ng/mL | 0.01 μM |
| 7 | YES | 1 ng/mL | 0.1 μM |
| 8 | YES | 1 ng/mL | 1 μM |

CSF-1 concentrations (pg/mL) vs time were plotted for each well using linear linear and log linear formats. Consumption was log linear over the first 4.9-hours. Excel was used to calculate a best fit linear equation describing CSF-1 consumption for each well through 4.9 hours and slopes were used to determine the relative rates of consumption.

### Results

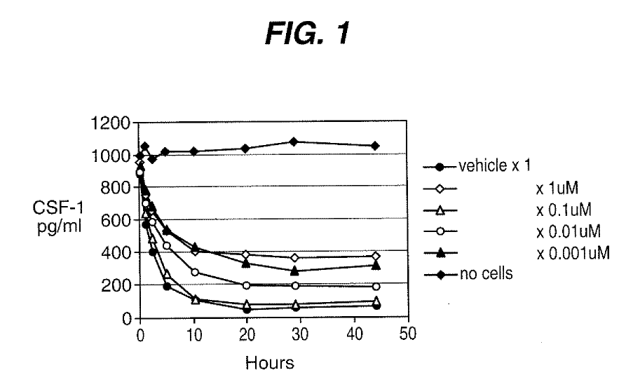

In the absence of cells, recombinant murine CSF-1 was stable under the current culture conditions (37° C., 5% CO₂, 1 mL/12 well plate well) for 44 hrs (FIG. 1). Condition media of BMDM cultured in the absence of recombinant CSF-1 contained no detectable CSF-1. Following addition of 1 ng/mL CSF-1 to BMDM, CSF-1 was consumed at a rate of about 37% per hour for the first several hours. COMPOUND 1 inhibited consumption of CSF-1 in a dose-dependent fashion. Rates of consumption were reduced 16, 56, 64, and 64% by 0.001, 0.01, 0.1, and 1 μM COMPOUND 1, respectively.

### Discussion

Herein three findings are made. Firstly, BMDM efficiently clear CSF-1 from culture media. A monolayer (˜60% confluent) of BMDM in a 12 well plate cleared CSF-1 at a rate of roughly 37% per hour when challenged with 1 ng of CSF-1 in 1 mL of media. Secondly, based on consumption rates, a portion (˜64%) of the clearance can be inhibited by COMPOUND 1. Half-maximal inhibition of consumption occurred at between 0.001 and 0.01 μM COMPOUND 1 consistent with the IC₅₀ of COMPOUND 1 for inhibition of CSF-1-induced BMDM proliferation (0.0026 μM). (data not shown) Consequently, this portion of the clearance is probably FMS kinase dependent. Thirdly, a portion (˜36%) is not inhibited even at 1 uM COMPOUND 1. Because this concentration is 100-1000 fold the IC₅₀ value, it can be assumed that FMS kinase is inhibited maximally, a conclusion that is consistent with the plateau effect on clearance achieved already at 0.1 μM COMPOUND 1. The uninhibited portion of CSF-1 clearance may be FMS kinase independent. This study does not distinguish FMS kinase independent from other possible FMS independent mechanisms. Overall, the in vivo data provide mechanistic bases for in vivo CSF-1 elevations caused by COMPOUND 1, i.e., direct inhibition of FMS kinase mediated CSF-1 clearance and indirect inhibition of clearance via the depletion of tissue macrophages.

No one has previously investigated the role of FMS kinase inhibitors in CSF consumption by macrophages. However, several studies have investigated the role of FMS kinase in FMS internalization and degradation. Carlberg et al. compared the rates of CSF-1 induced internalization of wild-type vs FMSA614 kinase dead mutant. During the first five minutes of CSF-1 exposure, approximately 85% of wild-type and 35% of kinase dead FMS were internalized. Thus, a significant portion (˜41%) of receptor internalization was FMS kinase independent, consistent with our CSF-1 consumption studies. More recently, Irvine et al. examined the effect of CYC10268, a newly described FMS kinase inhibitor, on CSF-1 induced FMS internalization on BMDM. A concentration of CYC10268 that markedly inhibited CSF-1 dependent FMS-phosphorylation failed to reduce FMS internalization at 20 minutes. In total, we conclude from these studies that FMS kinase-dependent and independent pathways exist for FMS-internalization and for CSF-1 clearance.

### Conclusions

Macrophages consume CSF-1. Nearly two-thirds of macrophage-mediated consumption can be inhibited by COMPOUND 1 and may therefore be FMS kinase dependent. These data provide one mechanistic basis for elevations in plasma CSF-1 following dose administration of COMPOUND 1. Slightly greater than one third of CSF-1 consumption were not inhibited by COMPOUND 1 suggesting a second, kinase-independent pathway of consumption.

### Example 2

Examination of the Tolerability and Biomarker Response of FMS Inhibitors (COMPOUND 2 & COMPOUND 1) in Rats

Groups of female Sprague Dawley rats (n=5) were orally administered 5 or 15 mg/kg of COMPOUND 2 or 40 mg/kg COMPOUND 1 twice per day for 5 consecutive days to characterize compound tolerability. Additional groups of rats, treated with vehicle or 40 mg/kg COMPOUND 1, were terminated one hour after the a.m. dose on Days 1 and 3 for interim analysis of various parameters. Post-mortem analysis of all treated animals included standard necropsy with liver histology evaluation as well as standard hematology and serology. None of the animals were found dead or were euthanized (moribund) prior to scheduled termination. None of the treatments affected body weight or the organ:body weight ratio of selected organs (i.e., liver, thymus, spleen & uterus). Both compounds were found to increase plasma concentrations of CSF-1 (4 to 6-fold) by Day 5. Both compounds, at the highest dose tested, decreased the number of macrophages in the uterus (up to ˜60% for COMPOUND 2) on Day 5 however, this parameter was highly variable. These compounds did not produce any overt dose-limiting toxicity.

The current study characterizes the tolerability and accompanying biomarker responses of two FMS/FLT3 tyrosine kinase inhibitors, COMPOUND 2 and COMPOUND 1.

COMPOUND 2 and COMPOUND 1- were synthesized. Test articles were stored dry at −20° C. Hydroxypropyl-b-cyclodextrin (CAS number 128446-35-5; Sigma) was prepared as a 20% (W:V) solution in water and served as the vehicle for test article preparation and as a vehicle control for the administration of test article.

COMPOUND 2 was prepared fresh daily as a clear solution in 20% HPbCD at 2.78 mg/ml and 8.33 mg/ml to deliver 5 and 15 mg per kg. COMPOUND 1 was prepared fresh daily as a clear solution in 20% HPbCD at 21.9 mg/ml to deliver 40 mg/kg.

TABLE 2
Allocation of Treatment Groups

| | | Treatment | Tissue Collection | |
|---|---|---|---|---|
| | | (po), bid | Day 1 & 3, 1 hr after a.m. dose | |
| Gp | N | Day 0, 1, 2, 3 & 4 | (5 rats in Groups 1 & 4) | Tissue Collection (early Day 5) |
| 1 | 15 | Vehicle | Whole blood, plasma, serum, weigh & | Whole blood, plasma, serum, |
| | | (20% HPCD) | discard liver, thymus, weigh and fix | weigh & fix liver, spleen, |
| | | | uterus, weigh and zap-freeze spleen | thymus & uterus and fix right |
| | | | | knee with femur and isolate left |
| | | | | femur on ice |
| 2 | 5 | COMPOUND 2 | None | Whole blood, plasma, serum, |
| | | (5 mg/kg) | | weigh & fix spleen, liver & |
| | | | | uterus and fix knee with femur |
| 3 | 5 | COMPOUND 2 | None | Whole blood, plasma, serum, |
| | | (15 mg/kg) | | weigh & fix spleen, liver & |
| | | | | uterus and fix knee with femur |
| 4 | 15 | COMPOUND 1 | Whole blood, plasma, serum, weigh & | Whole blood, plasma, serum, |
| | | (40 mg/kg) | discard liver, thymus, weigh and fix | weigh & fix spleen, liver & |
| | | | uterus, weigh and zap-freeze spleen | uterus and fix knee with femur |

On Day 0, the rats were ear tagged, randomized into the treatment groups (Groups 1 & 4: n=15, Groups 2 & 3: n=5), weighed and treated (po, bid) with either vehicle (20% HPbCD) or the test articles as delineated in Table 2 (Allocation of Treatment Groups).

On Days 1 & 3, one hour after a.m. dose, five rats from Groups 1 and 4 were euthanized using CO₂ asphyxiation and exsanguinated via cardiac puncture. Blood samples (˜500 μL) were transferred to separate ethylenediaminetetraacetic acid (EDTA) microtainers and a complete blood count (CBC) was conducted using the Advia 120 Hematology System (Bayer Diagnostics, Tarrytown, N.Y.). Additional blood samples were transferred to separate microtainers (with or without anticoagulant) and processed for biomarker (i.e., MCSF-1) and serologic (i.e., AST/ALT) analysis. The liver and thymus were weighed and discarded. The spleens were weighed and zap-frozen and the uteri (without ovaries attached) were weighed and fixed in 10% buffered formalin. The macrophage content of the uterus was determined immunohistochemically using a macrophage-specific (ED-1) antibody.

On Day 5, rats were euthanized using carbon dioxide and exsanguinated via cardiac puncture. Blood samples (˜500 μL) were collected and processed as described above for CBC, biomarker analysis and serology. The liver, spleen, thymus and uterus (without ovaries attached) were isolated, weighed and fixed in buffered formalin. The right knee with femur attached was isolated, trimmed and fixed in formalin. The left femur was isolated for determination of bone marrow cell counts. Liver histopathology was also conducted.

### Data Analysis

Differences between treated and control groups were analyzed statistically by ANOVA with a Dunnett's Multiple Comparison post-test. (p value; *:<0.05, **:<0.01).

### Results

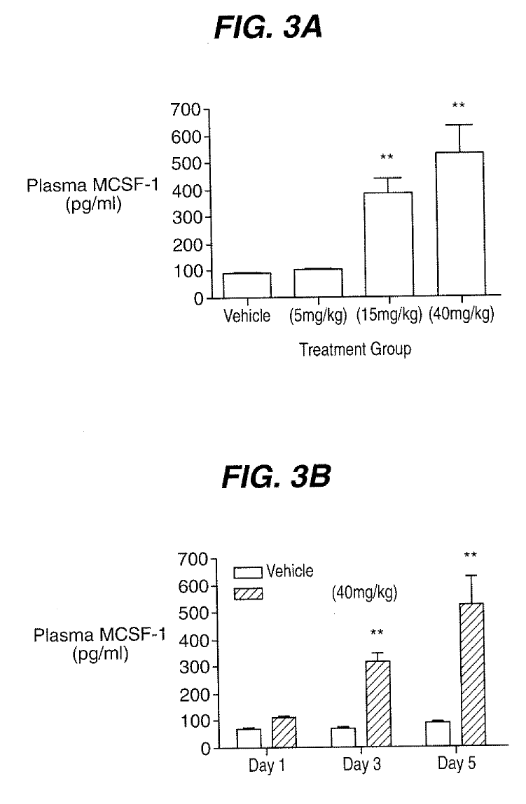

Treatment of rats with the FMS inhibitors COMPOUND 2 at 15 mg/kg and COMPOUND 1 at 40 mg/kg for 5 consecutive days resulted in increased plasma concentrations of CSF-1 that were 4 to 6-fold greater than control (FIG. 3A). COMPOUND 2 administered at a lower dose (i.e., 5 mg/kg) failed to significantly affect plasma concentrations of CSF-1. Examination of the plasma CSF-1 concentrations of rats treated with 40 mg/kg COMPOUND 1 at specific days throughout the study indicates that the elevated level of this factor observed on Day 5 develops gradually, with mean CSF-1 levels on Day 1, 3 and 5 being 112, 314 and 526 pg/ml, respectively (control values were ˜75 pg/ml, FIG. 3B).

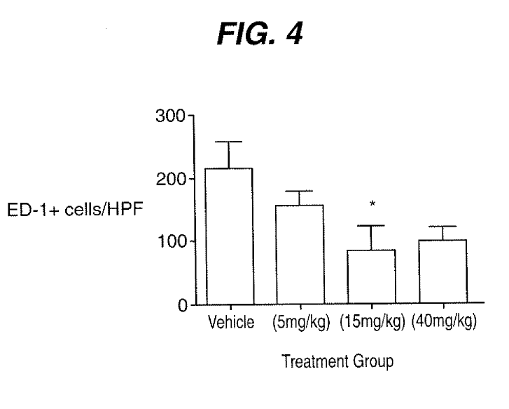

Treatment of rats with the FMS inhibitors was found to decrease the number of ED-1 positive macrophages in the uterus. Control uteri contained approximately 200 ED-1 positive cells per high power (microscopic) field, while treatment with COMPOUND 2 appeared to cause a dose dependent decrease (up to ˜60%) in the number of these cells/field (FIG. 4). Treatment with COMPOUND 1 also decreased the macrophage content of the uterus, however this parameter was highly variable with only COMPOUND 2 at 15 mg/kg inducing a significant effect (p-value; <0.05).

### Conclusions

Treatment of female Sprague Dawley rats for 5 consecutive days (po, bid) with 5 or 15 mg/kg of COMPOUND 2 or 40 mg/kg COMPOUND 1 did not have an observable effect on the appearance, behavior, body weight or the organ:body weight ratio of specific organs including liver, spleen and thymus. Both compounds were found to increase plasma concentrations of CSF-1 above control levels by Day 5. Both compounds at the highest dose tested, decreased the number of macrophages in the uterus on Day 5 however, this parameter was highly variable. These compounds did not produce any overt dose-limiting toxicity.

This biomarker may be used in accordance with the invention to assess response to FMS treatments in patients. For example, inhibition or lack of inhibition of FMS can be determined in order to predict a clinical response.

### Example 3

Pharmacodynamic activity in rats was determined to identify appropriate doses for efficacy studies. Circulating CSF-1 is cleared by sinusoidal macrophages when bound and internalized by FMS in a process that is partly dependent on FMS kinase activity. CSF-1 levels rise when FMS is inhibited, or when FMS inhibition reduces the number/function of macrophages. Uterine macrophages are short-lived and FMS-dependent. Quantitation of uterine macrophage density and plasma CSF-1 levels thereby provided pharmacodynamic endpoints measurable in rats following 4 days of dosing.

Method: Female Sprague Dawley Rats (150 to 200 g BW) from Charles River were administered vehicle or COMPOUND 3 at 10 and 40 mg/kg p.o. bid for 4 days. On the fifth day, a final dose was given and the animals were sacrificed 2 hrs later. Rats were euthanized using CO₂ asphyxiation and exsanguinated via cardiac puncture. An aliquot of blood (1000 mL) was transferred to an EDTA tube and processed for plasma and for determination of plasma compound and CSF-1 biomarker levels. In addition, an aliquot of blood (˜2 mL) was transferred to a microfuge tube that did not contain anticoagulant and serum was prepared by centrifugation following incubated at room temperature for 1 hour.

Spleens, livers and uteri (without ovaries) were isolated and weighed and uteri were fixed in formalin. Uteri sections were stained for ED1 and ED-1 stained area (arbitrary units) at 200× magnification was determined using Image-Pro Plus software.

Results: Mean plasma levels of COMPOUND 3 two hours following the last dose of 10 and 40 mg/kg were 2537 ng/mL and 5370 ng/mL, respectively. CSF-1 levels were elevated 5.5-fold in rats dosed with 40 mg/kg COMPOUND 3 and 4.5-fold in rats dosed with 10 mg/kg COMPOUND 3 (Table 3). Uterine macrophages were depleted by 51 and 81% at 10 and 40 mg/kg, respectively. Macrophages present in rats dosed with 40 mg/kg did not have the normal reticulated morphology; ED-1 staining was either localized to large round cells or to what appeared to be cell fragments. Based on the elevation in CSF-1 and the reduction of uterine macrophages 10 and 40 mg/kg were identified as doses with robust pharmacodynamic activity.

TABLE 3
Plasma drug levels and pharmacodynamic biomarkers in rats dosed 4 days with
COMPOUND 3.

| | | Compound, | Mean | CSF-1, | | Uterine ED1 | Mean |
|---|---|---|---|---|---|---|---|
| Animal # | Treatment | ng/ml | (SEM) | pg/ml* | Mean (SEM) | area/HPF | (SEM) |
| 227 | vehicle | | | 149 | 127 | 13190 | 9808 |
| 226 | | | | 122 | (9) | 8089 | (1350) |
| 221 | | | | 117 | | 9525 | |
| 230 | | | | 118 | | 8428 | |
| 205 | COMPOUND 3 | 2730 | 2537 | 651 | 570 | 4919 | 4855 |
| 212 | 10 mg/kg | 2080 | (285) | 482 | (60) | 7609 | (1971) |
| 231 | | 2800 | | 577 | | 2037 | |
| 233 | COMPOUND 3 | 5320 | 5370 | 548 | 698 | 929 | 1836 |
| 202 | 40 mg/kg | 5060 | (240) | 746 | (94) | 1721 | (686) |
| 209 | | 5730 | | 801 | | 2858 | |

*Values were determined using a mouse CSF-1 ELISA (R&D Systems). The exact level of cross-reactivity to rat CSF-1 is unknown. The values probably underestimate true concentrations. For comparative purpose, this ELISA detects ~1000 pg/mL CSF-1 in normal mouse plasma.

The structure of COMPOUND 3 is reproduced below:

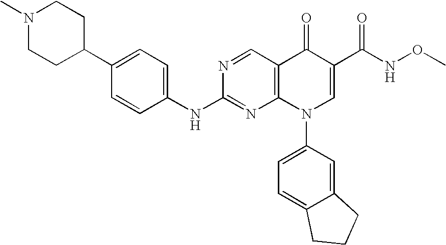

Compounds like COMPOUND 3 and their use are disclosed in U.S. Application Nos. 200700605771A1 and 20070060578A1.

### Example 4

TABLE 4
Plasma drug levels and pharmacodynamic biomarkers
in rats dosed 4 days with COMPOUND 4.

| | | Plasma | | |
|---|---|---|---|---|
| | Animal | compound, | Plasma | Uterine ED1 |
| Treatment | # | ng/ml | CSF-1, pg/ml | area/HPF |
| Vehicle (20% | 204 | | 117 | 1677 |
| solutol) | 203 | | 101 | 2417 |
| | 212 | | 134 | 2064 |
| | 207 | | 133 | 3329 |
| | 226 | | 136 | 798 |
| | Mean | | 124 | 2057 |
| COMPOUND 4, | 231 | 1070 | 152 | 1003 |
| 10 mpk | 209 | 968 | 165 | 1233 |
| | 247 | 1560 | 139 | 1174 |
| | 223 | 1790 | 157 | 1021 |
| | 248 | 1680 | 184 | 3075 |
| | Mean | 1414 (186) | 159* | 1501 |
| COMPOUND 4, | 211 | 2510 | 211 | 1419 |
| 20 mpk | 232 | 2670 | 163 | 1995 |
| | 217 | 2320 | 196 | 1164 |
| | 225 | 2470 | 238 | 1560 |
| | 221 | 3210 | 249 | 1244 |
| | Mean | 2636 (172) | 211** | 1476 |
| COMPOUND 4, | 230 | 5670 | 540 | 681 |
| 50 mpk | 213 | 6090 | 696 | 1431 |
| | 250 | 4800 | 506 | 2955 |
| | 240 | 9390 | 585 | 585 |
| | 245 | 7110 | 541 | 94 |
| | Mean | 6612 (881) | 574** | 1149 |

*Values were determined using a mouse CSF-1 ELISA (R&D Systems). The level of cross-reactivity to rat CSF-1 is unknown. The values probably underestimate true concentrations. For comparative purpose, this ELISA detects ca. 1000 pg/ml CSF-1 in normal mouse plasma. ADME07-334
*p < 0.05 vs. Vehicle.
**p < 0.005 vs. Vehicle.

Results: Mean plasma levels of COMPOUND 4 2 hours following the last dose of 10, 20, and 50 mpk were 1414 ng/ml, 2636 ng/ml, and 6612 ng/ml, respectively. CSF-1 levels were elevated significantly 28, 79 and 363% in rats dosed with 10, 20 and 50 mpk, respectively (Table 4). Uterine macrophages were depleted by 27%, 28%, and 44% at 10, 20, and 50 mpk COMPOUND 4, respectively. Based on these results, 10 mg/kg was identified as a dose with a threshold level of activity for these endpoints.

The structure of COMPOUND 4 is reproduced below:

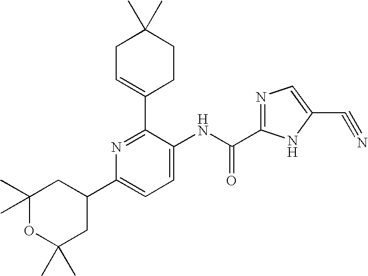

COMPOUND 4 and its use are disclosed in U.S. Application Ser. No. 60/980,263, filed Oct. 17, 2007.

### Example 5

The safety and tolerability of single ascending, oral doses of COMPOUND 1 in healthy male subjects was assessed. Blood samples were collected on Day 1 predose and at 2, 4, 8, 12, 24 and 48 hours postdose and assessed for biomarker. Analysis of the biomarker was performed for all subjects receiving at least one dose of COMPOUND 1 or placebo as set forth in Table 5.

TABLE 5
Part 1
Dosing Scheme

| | COMPOUND 1 | Placebo |
|---|---|---|
| | Treatment Group | Treatment Group |
| Cohort | N = 6 | N = 2 |
| 1 | 150 mg | placebo |
| 2 | 300 mg | placebo |
| 3 | 600 mg | placebo |
| 4 | 1200 mg | placebo |

4 mL venous blood samples were collected on Day 1, within 60 minutes (predose), 2, 4, 8, 12, 24, and 48 hours postdose and 3 mL on Day 1 predose, 2 and 8 hours postdose for assessment of the effect of COMPOUND 1 on plasma CSF-1.

Plasma CSF-1 levels were measured using R&D Systems Human CSF-1 ELISA. Samples were diluted 1:5 for assay. Assay range for standards is 31-2000 pg/mL. Maximum measurable plasma concentration is ˜10000 pg/ml. Current maximum measured concentration ˜4000 pg/ml. The results are shown in the following tables.

Fold Change from Time 0 150 mg dose

| Time Pt. | 3001 | 3002 | 3003 | 3004 | 3006 | 3007 | 3008 |
|---|---|---|---|---|---|---|---|
| 0 | 1.0 | 1.0 | 1.0 | 1.0 | 1.0 | 1.0 | 1.0 |
| 2 | 1.8 | 2.5 | 2.0 | 0.8 | 0.8 | 1.8 | 0.9 |
| 4 | 2.2 | 1.8 | 1.9 | 0.8 | 0.9 | 1.9 | 0.8 |
| 8 | 1.4 | 2.5 | 1.6 | 1.0 | 0.8 | 1.7 | 0.7 |
| 12 | 1.5 | 1.6 | 2.4 | 0.9 | 0.8 | 1.7 | 0.7 |
| 24 | 1.4 | 1.3 | 1.2 | 2.0 | 0.9 | 1.8 | 0.7 |
| 48 | 1.2 | 1.1 | 1.0 | 0.8 | 1.8 | 1.4 | 0.6 |

Fold Change from Time 0 300 mg dose

| Time Pt. | 3009 | 3010 | 3011 | 3012 | 3013 | 3014 | 3015 | 3016 |
|---|---|---|---|---|---|---|---|---|
| 0 | 1.0 | 1.0 | 1.0 | 1.0 | 1.0 | 1.0 | 1.0 | 1.0 |
| 2 | 4.6 | 0.8 | 3.3 | 2.7 | 0.9 | 5.2 | 3.4 | 1.8 |
| 4 | 4.4 | 1.4 | 3.0 | 4.4 | 0.7 | 6.8 | 4.5 | 2.6 |
| 8 | 3.8 | 0.9 | 3.4 | 3.0 | 1.5 | 6.4 | 4.1 | 1.9 |
| 12 | 2.6 | 0.8 | 2.2 | 3.8 | 0.9 | 4.5 | 3.0 | 1.4 |
| 24 | 1.9 | 0.9 | 1.5 | 1.5 | 1.7 | 2.1 | 1.5 | 1.1 |
| 48 | 2.3 | 1.0 | 1.4 | 1.4 | 0.9 | 2.2 | 1.2 | 0.8 |

Fold Change From Time 0 600 mg dose

| Time Pt. | 3017 | 3018 | 3019 | 3020 | 3021 | 3022 | 3023 | 3024 |
|---|---|---|---|---|---|---|---|---|
| 0 | 1.0 | 1.0 | 1.0 | 1.0 | 1.0 | 1.0 | 1.0 | 1.0 |
| 2 | 7.5 | 0.7 | 2.9 | 5.0 | 0.9 | 6.0 | 4.8 | 2.3 |
| 4 | 8.7 | 1.2 | 5.2 | 5.1 | 0.8 | 8.8 | 6.9 | 3.1 |
| 8 | 9.4 | 1.2 | 4.4 | 5.4 | 0.9 | 10.1 | 7.9 | 3.3 |
| 12 | 5.9 | 0.7 | 6.0 | 4.9 | 0.9 | 6.6 | 4.9 | 3.0 |
| 24 | 2.8 | 0.7 | 2.3 | 2.4 | 2.4 | 3.5 | 2.7 | 1.8 |
| 48 | 2.2 | 0.6 | 1.5 | 1.9 | 0.8 | 3.0 | 1.5 | 1.4 |

Fold Change from Baseline 1200 mg dose

| | 3025 | 3026 | 3027 | 3028 | 3029 | 3030 | 3031 | 3032 |
|---|---|---|---|---|---|---|---|---|
| 0 | 1.0 | 1.0 | 1.0 | 1.0 | 1.0 | 1.0 | 1.0 | 1.0 |
| 2 | 5.4 | 4.9 | 0.8 | 4.4 | 6.8 | 4.4 | 0.5 | 3.5 |
| 4 | 8.9 | 8.4 | 1.0 | 7.6 | 9.5 | 5.9 | 0.4 | 5.3 |
| 8 | 9.9 | 9.8 | 1.0 | 11.3 | 13.4 | 6.9 | 0.4 | 7.8 |
| 12 | 9.2 | 5.2 | 2.1 | 10.5 | 8.8 | 5.8 | 0.4 | 6.0 |
| 24 | 4.7 | 1.4 | 1.0 | 6.1 | 2.9 | 3.2 | 0.4 | 1.8 |
| 48 | 2.7 | 1.0 | 0.9 | 2.7 | 3.3 | 2.4 | 0.6 | 1.6 |

While the foregoing specification teaches the principles of the present invention, with examples provided for the purpose of illustration, it will be understood that the practice of the invention encompasses all of the usual variations, adaptations and modifications as come within the scope of the following claims and their equivalents

### REFERENCES

1. Bartocci A, Mastrogiannis D S, Migliorati G, Stockert R J, Wolkoff W W, Stanley E R. Macrophages specifically regulate the concentration of their own growth factor in the circulation. Proc Natl Acad Sci USA; 1987; 84:6179-83.
2. Carlberg K, Tapley P, Haystead C, Rohrschneider L. The role of kinase activity and the kinase insert region in ligand induced internalization and degradation of the c fms protein. EMBO J. 10(4) (1991) 877-83.
3. Irvine K M, Burns C J, Wilks, A F, Su S, Hume D A, Sweet M J. A CSF-1 receptor kinase inhibitor targets effector functions and inhibits pro inflammatory cytokine production from murine macrophage populations. The FASEB J (2006) 20: 1921-3.
4. Dai X M, Ryan G R, Hapel A J, Dominguez M G, Russell R G, Kapp S, Sylvestre V, Stanley E R. Targeted disruption of the mouse colony-stimulating factor 1 receptor gene results in osteopetrosis, mononuclear phagocyte deficiency, increased primitive progenitor cell frequencies, and reproductive defects. Blood. 99 (2002) 111-20.

1. A biomarker predictive of a response of cells to treatment with a drug that inhibits FMS activity.

2. The biomarker of claim 1, wherein the biomarker is predictive of a response of a human subject or other animal to treatment with a drug that inhibits FMS activity.

3. A method for predicting whether a drug is capable of inhibiting FMS activity in a cell, comprising the steps of: a) obtaining a first sample of cells prior to administration of said drug and a second sample of cells after administration of said drug; b) determining the level of CSF-1 in said first sample of cells and said second sample of cells; c) comparing the levels of CSF-1; and d) correlating any change of CSF-1 to said drugs' ability or inability to inhibit FMS activity in said cells.

4. The method of claim 3, wherein the method is for predicting whether a drug is capable of inhibiting FMS activity in a human subject or other animal, the steps of: a) obtaining a first sample of serum or plasma prior to dose administration of said drug and a second sample of serum or plasma after dose administration of said drug; b) determining the level of CSF-1 in said first sample of serum or plasma and said second sample of serum or plasma; c) comparing the levels of CSF-1; and d) correlating any change of CSF-1 to said drugs' ability or inability to inhibit FMS activity in said human subject or other animal.

5. A method of screening for candidate drugs capable of inhibiting the activity of FMS, comprising: a) contacting a test drug with a sample of cells; and b) selecting as candidate drugs those test drugs that increase the level of CSF-1 in said sample of cells.

6. The method of claim 5, wherein the method is a method of screening for candidate drugs capable of inhibiting the activity of FMS in a human subject or other animal, comprising: a) contacting a test drug with a human subject or other animal; and b) selecting as candidate drugs those test drugs that increase the serum or plasma level of CSF-1 in said human subject or other animal.
